(19) United States
(12) Patent Application Publication
(10) Pub. No.: US 2007/0011829 A1
(43) Pub. Date: Jan. 18, 2007

(54) Novel double para-phenylenediamines joined by a linkage comprising an atom chosen from sulphur and nitrogen and method for dyeing keratinous fibers

(76) Inventors: Stephane Sabelle, Paris (FR); Eric Metais, St. Leu la Foret (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/476,824

(22) Filed: Jun. 29, 2006

Related U.S. Application Data
(60) Provisional application No. 60/698,933, filed on Jul. 14, 2005.

(30) Foreign Application Priority Data
Jun. 29, 2005 (FR) 0551808

(51) Int. Cl. A61K 8/00

(52) U.S. Cl. 8/405

(57) ABSTRACT

Disclosed herein is a novel family of double para-phenylenediamines joined by a linkage comprising an atom chosen from sulphur and nitrogen. Also disclosed herein is a dyeing composition comprising at least one double para-phenylenediamine joined by a linkage comprising an atom chosen from sulphur and nitrogen. Further disclosed herein is a method for dyeing keratin fibers comprising applying such a composition to the keratin fibers. A multi-compartment device comprising at least one first compartment containing at least one dyeing composition of the present disclosure and at least one second compartment containing at least one oxidizing composition is also disclosed herein.

This application claims benefit of U.S. Provisional Application No. 60/698,933, filed Jul. 14, 2005, the contents of which are incorporated herein by reference. This application also claims benefit of priority under 35 U.S.C. § 119 to French Patent Application No. FR 05 51808, filed Jun. 29, 2005, the contents of which are also incorporated herein by reference.

Disclosed herein is a novel family of double para-phenylenediamines joined by a linkage comprising an atom chosen from sulphur and nitrogen atoms. Also disclosed herein is a dyeing composition comprising at least one double para-phenylenediamine joined by a linkage comprising an atom chosen from sulphur and nitrogen atoms. Further disclosed herein is a method for dyeing keratin fibers comprising applying such a composition to the keratin fibers. Still further disclosed herein is a multi-compartment device comprising at least one first compartment containing at least one dyeing composition of the present disclosure and at least one second compartment containing at least one oxidizing composition.

It is common practice to dye keratin fibers, for example, human hair, with dyeing compositions comprising oxidation dye precursors, generally called oxidation bases, such as ortho- or para-phenylenediamines, ortho- or para-aminophenols, and heterocyclic compounds. These oxidation bases are colorless or weakly colored compounds, which, when combined with oxidizing products can give rise, by a process of oxidative condensation, to colored compounds.

It is also known that it is possible to vary the shades obtained with these oxidation bases by combining them with couplers or dyeing modifiers, the latter being chosen, for example, from aromatic meta-diaminobenzenes, meta-aminophenols, meta-diphenols, and certain heterocyclic compounds, such as indole compounds and pyridine compounds.

The great variety of the molecules available as oxidation bases and couplers may allow a rich palette of colors to be obtained.

The so-called “permanent” dyeing obtained using these oxidation dyes ideally satisfies at least one of a number of requirements. For example, the dye ideally does not present any problems of a toxicological nature, makes it possible to obtain the color shades of the desired intensity, has good resistance to external agents, such as light, weather, washing, permanent waving, sweat, and/or rubbing, provides coverage of white hair, and displays minimum selectivity, i.e., ensures that the smallest possible differences in coloration are obtained along the same keratin fiber, which generally is differently sensitized (i.e., damaged) between its tip and its root.

The inventors have discovered novel oxidation bases which may be capable of dyeing keratin fibers in a variety of strong, aesthetic shades with low selectivity, and which may be resistant to the various aggressive factors to which the fibers may be subjected, such as light, sweat, and/or shampoos.

Thus, disclosed herein is a novel family of double para-phenylenediamines of formula (I) and the addition salts thereof: 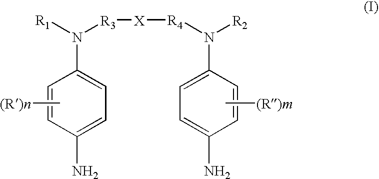
wherein:
X is chosen from:
   sulphur, and
   —NR₅— radicals,
R₃ and R₄, which may be identical or different, are chosen from C₂-C₆ alkylene radicals,
R₁ and R₂, which may be identical or different, are chosen from:
   hydrogen,
   linear and branched C₁-C₆ alkyl radicals, and
   linear and branched C₁-C₆ alkyl radicals substituted by at least one radical chosen from hydroxy, C₁-C₄ alkoxy, amino, C₁-C₄ monoalkylamino, and C₁-C₄ dialkylamino radicals,
R′ and R″, which may be identical or different, are chosen from:
   C₁-C₆ alkyl radicals,
   C₁-C₆ alkoxy radicals,
   C₁-C₆ hydroxy-alkoxy radicals,
   C₁-C₆ alkoxy(C₁-C₆)alkyl radicals, and
   C₁-C₆ mono- and poly-hydroxy alkyl radicals,
R₅ is chosen from:
   hydrogen,
   linear and branched C₁-C₁₂ alkyl radicals,
   tosyl radicals,
   mesyl radicals, and
   benzene rings optionally functionalized with at least one group chosen from C₁-C₄ alkyl groups and 3-carboxypropyl groups, and
n and m, which may be identical or different, are chosen from integers ranging from 0 to 4.

The composition of the present invention may make it possible to obtain very strong coloration of keratin fibers of low selectivity, which may be resistant to various external factors, for example, light.

Also disclosed herein are compositions for dyeing keratin fibers, for example, human keratin fibers such as the hair, comprising at least one para-phenylenediamine of formula (I).

Further disclosed herein is a method for dyeing keratin fibers comprising applying a dyeing composition of the present disclosure to the keratin fibers, for instance, human keratin fibers, such as the hair. Still further disclosed herein is a multi-compartment device or dyeing “kit” comprising at least one first compartment containing at least one dyeing composition of the present disclosure and at least one second compartment containing at least one oxidizing agent.

Compounds

Para-phenylenediamine Compounds of Formula (I)

Examples of para-phenylenediamine compounds of formula (I) include, but are not limited to:

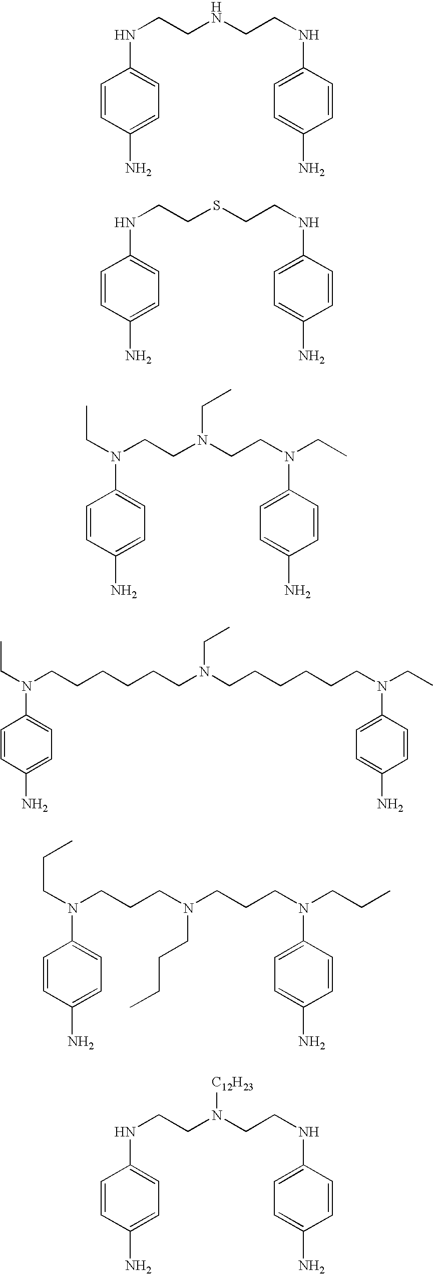

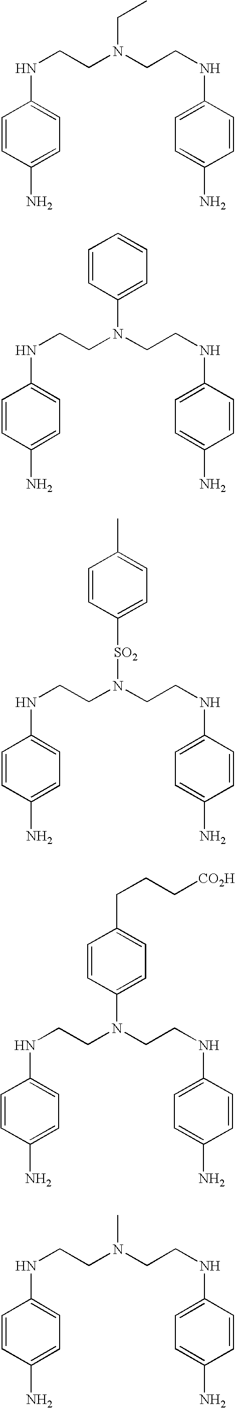

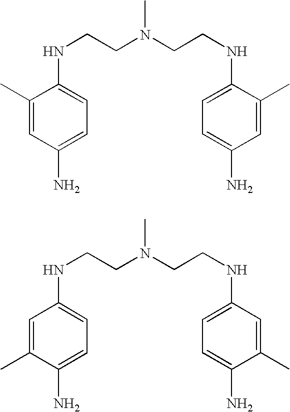

According to one embodiment of the present disclosure, in formula (I), R₁ and R₂, which may be identical or different, are chosen from hydrogen and optionally substituted C₁-C₄ alkyl groups. In another embodiment, R₃ and R₄, which may be identical or different, may be chosen from C₂-C₃ alkylene radicals. In a further embodiment, n and m, which may be identical or different, are integers ranging from 0 to 1. In yet another embodiment, R₅ may be chosen from hydrogen and C₁-C₆ alkyl radicals.

The addition salts may be chosen, for example, from acid addition salts, such as hydrochloric acid, hydrobromic acid, sulphuric acid, citric acid, succinic acid, tartaric acid, lactic acid, para-toluene-sulphonic acid, benzene-sulphonic acid, phosphoric acid, and acetic acid addition salts.

They may also be in the form of solvates, for example, hydrates and solvates of linear and branched alcohols, such as ethanol and isopropanol.

The para-phenylenediamines of formula (I) according to the present disclosure may be prepared according to conventional methods of synthesis (see, for example, German Patent Application No. 101 44 226 A).

For example, the para-phenylenediamines of formula (I) may be synthesized according to the following reaction scheme:

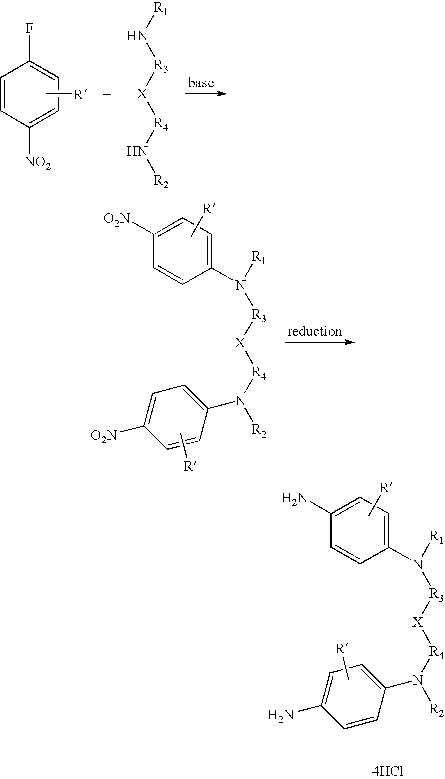

The first stage in the synthesis is a nucleophilic substitution of a diamine on a derivative of para-fluoro-nitrobenzene, a stage suggested, for example, by the publications Synthesis 12:, 1147-1148 (1990) and Synth. Commun. 20 (22), 3537-3545 (1990). The second stage is a conventional reduction stage, and may be, for example, a hydrogenation reaction by heterogeneous catalysis in the presence of a catalyst chosen from Pd/C, Pd(II)/C, and Raney Nickel, or a reduction reaction by a metal, for example, by zinc, iron, tin, and the like (see, for example, J. March, Advanced Organic Chemistry, 4th ed., 1992, Wiley Interscience; M. Hudlicky, Reduction in Organic Chemistry, 1983, Ellis Horwood Series Chemical Science).

Also disclosed herein are nitrogen-containing compounds of formula (II) which may be used as intermediate compounds for obtaining para-phenylenediamines of formula (I): 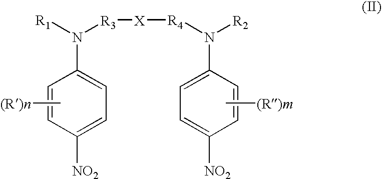
wherein R₁, R₂, X, R′, R″, R₃, R₄, n, and m are defined above in the context of formula (I), and
with the proviso that the compound of formula (II) is not chosen from the following compounds: 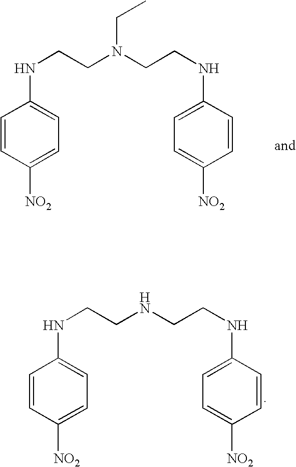
Dyeing Composition

Further disclosed herein is a dyeing composition comprising, in a medium suitable for dyeing, at least one para-phenylenediamine oxidation base chosen from compounds of formula (I) and the addition salts thereof.

The at least one para-phenylenediamine oxidation base may be present in the composition in an amount ranging from 0.0001 wt. % to 20 wt. % relative to the total weight of the composition, for example, from 0.01 wt. % to 10 wt. %.

Oxidation Coupler

According to one embodiment, the composition of the present disclosure may comprise at least one oxidation coupler.

Examples of oxidation couplers include, but are not limited to, meta-phenylenediamines, meta-aminophenols, meta-diphenols, naphthalenic couplers, heterocyclic couplers, and the addition salts thereof.

Suitable oxidation couplers may include, for example, 2-methyl-5-aminophenol, 5-N-(β-hydroxyethyl)amino-2-methylphenol, 6-chloro-2-methyl-5-aminophenol, 3-aminophenol, 1,3-dihydroxybenzene (or resorcinol), 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diamino-benzene, 1,3-bis-(2,4-diaminophenoxy)propane, 3-ureido-aniline, 3-ureido-1-dimethylaminobenzene, sesamol, 1-β-hydroxyethylamino-3,4-methylene-dioxybenzene, α-naphthol, 2-methyl-1-naphthol, 6-hydroxy-indole, 4-hydroxy-indole, 4-hydroxy-N-methyl indole, 2-amino-3-hydroxypyridine, 6-hydroxybenzomorpholine, 3,5-diamino-2,6-dimethoxypyridine, 1-N-(β-hydroxyethyl)amino-3,4-methylene dioxybenzene, 2,6-bis-(β-hydroxyethylamino)toluene, and the addition salts thereof.

The at least one oxidation coupler may be present in the composition in an amount ranging from 0.0001 to 20 wt. %, for example, from 0.005 to 6 wt. %, relative to the total weight of the composition.

Additional Oxidation Bases

The composition according to the present disclosure may also comprise at least one additional oxidation base different from the oxidation bases of formula (I).

The oxidation bases may be chosen, for example, from para-phenylenediamines, bis-phenylalkylenediamines, para-aminophenols, ortho-aminophenols, heterocyclic bases, and the addition salts thereof.

Examples of para-phenylenediamines include, but are not limited to: para-phenylenediamine, para-toluenediamine, 2-chloro-para-phenylenediamine, 2,3-dimethyl para-phenylenediamine, 2,6-dimethyl para-phenylenediamine, 2,6-diethyl para-phenylenediamine, 2,5-dimethyl para-phenylenediamine, N,N-dimethyl para-phenylenediamine, N,N-diethyl para-phenylenediamine, N,N-dipropyl para-phenylenediamine, 4-amino-N,N-diethyl-3-methyl aniline, N,N-bis-(β-hydroxyethyl)para-phenylenediamine, 4-N,N-bis-(β-hydroxyethyl)amino-2-methyl aniline, 4-N,N-bis(β-hydroxyethyl)amino-2-chloro-aniline, 2-β-hydroxyethyl para-phenylenediamine, 2-fluoro para-phenylenediamine, 2-isopropyl para-phenylenediamine, N-(β-hydroxypropyl)para-phenylenediamine, 2-hydroxymethyl para-phenylenediamine, N,N-dimethyl-3-methyl para-phenylenediamine, N,N-(ethyl, β-hydroxyethyl)para-phenylenediamine, N-(β,γ-dihydroxypropyl) para-phenylenediamine, N-(4′-aminophenyl)para-phenylenediamine, N-phenyl para-phenylenediamine, 2-β-hydroxyethyloxy para-phenylenediamine, 2-α-acetylaminoethyloxy para-phenylenediamine, N-(β-methoxyethyl)para-phenylenediamine, 2-thienyl para-phenylenediamine, 2-β-hydroxyethylamino-5-aminotoluene, 3-hydroxy-1-(4′-aminophenyl)pyrrolidone, 6-(4-amino-phenylamino)-hexan-1-ol, and the acid addition salts thereof.

Suitable para-phenylenediamines may include, for example, para-phenylenediamine, para-toluenediamine, 2-isopropyl para-phenylenediamine, 2-β-hydroxyethyl para-phenylenediamine, 2-β-hydroxyethyloxy para-phenylenediamine, 2,6-dimethyl para-phenylenediamine, 2,6-diethyl para-phenylenediamine, 2,3-dimethyl para-phenylenediamine, N,N-bis-(β-hydroxyethyl)para-phenylenediamine, 2-chloro para-phenylenediamine, 2-β-acetylaminoethyloxy para-phenylenediamine, 6-(4-amino-phenylamino)-hexan-1-ol, and the acid addition salts thereof.

Non-limiting examples of bis-phenylalkylenediamines include: N,N′-bis-(β-hydroxyethyl) N,N′-bis-(4′-aminophenyl) 1,3-diamino-propanol, N,N′-bis(β-hydroxyethyl) N,N′-bis(4′-aminophenyl) ethylenediamine, N,N′-bis-(4′-aminophenyl)tetramethylenediamine, N,N′-bis-(β-hydroxyethyl) N,N′-bis-(4′-aminophenyl)tetramethylenediamine, N,N′-bis-(4′-amino-3′-methylphenyl)tetramethylenediamine, N,N′-bis-(ethyl) N,N′-bis-(4′-amino, 3′-methylphenyl) ethylenediamine, 1,8-bis-(2,5-diamino-phenoxy)-3,6-dioxaoctane, and the acid addition salts thereof.

Para-aminophenols suitable for use in accordance with the present disclosure may include, for example: para-aminophenol, 4-amino-3-methylphenol, 4-amino-3-fluorophenol, 4-amino-2-chlorophenol, 4-amino-3-chlorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethyl aminomethyl)phenol, 4-amino-2-fluorophenol, 4-amino-2,6-dichlorophenol, 4-amino-6[((5′-amino-2′-hydroxy-3′-methyl)phenyl)methyl]-2-methylphenol, bis(5′-amino-2′-hydroxy)phenylmethane, and the acid addition salts thereof.

Examples of ortho-aminophenols include, but are not limited to: 2-aminophenol, 2-amino-5-methylphenol, 2-amino-6-methylphenol, 5-acetamido-2-aminophenol, 5-[(2-hydroxyethyl)amino]2-methylphenol, and the acid addition salts thereof.

Suitable heterocyclic bases include, for example: pyridine derivatives, pyrimidine derivatives, and pyrazole derivatives.

Non-limiting examples of pyridine derivatives may include the compounds described, for example, in British Patent Nos. 1 026 978 and 1 153 196, such as 2,5-diamino pyridine, 2-(4-methoxyphenyl)amino-3-amino pyridine, 3,4-diamino pyridine, and the acid addition salts thereof.

Other examples of suitable pyridine oxidation bases include, but are not limited to, 3-amino pyrazolo-[1,5-a]-pyridines and the addition salts thereof, described, for example, in French Patent Application No. 2 801 308. Examples of these compounds include, but are not limited to, pyrazolo[1,5-a]pyridin-3-ylamine; 2-acetylamino pyrazolo-[1,5-a]pyridin-3-ylamine; 2-morpholin-4-yl-pyrazolo[1,5-a]pyridin-3-ylamine; 3-amino-pyrazolo[1,5-a]pyridine-2-carboxylic acid; 2-methoxy-pyrazolo[1,5-a]pyridin-3-ylamine; (3-amino-pyrazolo[1,5-a]pyridin-7-yl)-methanol; 2-(3-amino-pyrazolo[1,5-a]pyridin-5-yl)-ethanol; 2-(3-amino-pyrazolo[1,5-a]pyridin-7-yl)-ethanol; (3-amino-pyrazolo[1,5-a]pyridin-2-yl)-methanol; 3,6-diamino-pyrazolo[1,5-a]pyridine; 3,4-diamino-pyrazolo[1,5-a]pyridine; pyrazolo[1,5-a]pyridine-3,7-diamine; 7-morpholin-4-yl-pyrazolo[1,5-a]pyridin-3-ylamine; pyrazolo[1,5-a]pyridine-3,5-diamine; 5-morpholin-4-yl-pyrazolo[1,5-a]pyridin-3-ylamine; 2-[(3-amino-pyrazolo[1,5-a]pyridin-5-yl)-(2-hydroxyethyl)-amino]-ethanol; 2-[(3-amino-pyrazolo[1,5-a]pyridin-7-yl)-(2-hydroxyethyl)-amino]-ethanol; 3-amino-pyrazolo[1,5-a]pyridin-5-ol; 3-amino-pyrazolo[1,5-a]pyridin-4-ol; 3-amino-pyrazolo[1,5-a]pyridin-6-ol; 3-amino-pyrazolo[1,5-a]pyridin-7-ol; and the acid addition salts thereof.

Pyrimidine derivatives suitable for use in accordance with the present disclosure may include the compounds described, for example, in German Patent No. 23 59 399; Japanese Patent Application No. 88-169571; Japanese Patent No. 05-63124; European Patent No. 0 770 375; and International Patent Application Publication No. WO 96/15765, such as 2,4,5,6-tetra-aminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine, and the addition salts and tautomeric forms thereof, when a tautomeric equilibrium exists.

Examples of pyrazole derivatives may include, but are not limited to, the compounds described in German Patent Nos. 38 43 892 and 41 33 957, International Patent Application Publication Nos. WO 94/08969 and WO 94/08970, French Patent Application No. 2 733 749, and German Patent Application No. 195 43 988, such as 4,5-diamino-1-methyl pyrazole, 4,5-diamino-1-(β-hydroxyethyl) pyrazole, 3,4-diamino pyrazole, 4,5-diamino-1-(4′-chlorobenzyl) pyrazole, 4,5-diamino-1,3-dimethyl pyrazole, 4,5-diamino-3-methyl-1-phenyl pyrazole, 4,5-diamino-1-methyl-3-phenyl pyrazole, 4-amino-1,3-dimethyl-5-hydrazino pyrazole, 1-benzyl-4,5-diamino-3-methyl pyrazole, 4,5-diamino-3-tert-butyl 1-methyl pyrazole, 4,5-diamino-1-tert-butyl 3-methyl pyrazole, 4,5-diamino-1-(β-hydroxyethyl)-3-methyl pyrazole, 4,5-diamino-1-ethyl-3-methyl pyrazole, 4,5-diamino-1-ethyl-3-(4′-methoxyphenyl) pyrazole, 4,5-diamino-1-ethyl-3-hydroxymethyl pyrazole, 4,5-diamino-3-hydroxymethyl-1-methyl pyrazole, 4,5-diamino-3-hydroxymethyl-1-isopropyl pyrazole, 4,5-diamino-3-methyl-1-isopropyl pyrazole, 4-amino-5-(2′-aminoethyl)amino-1,3-dimethyl pyrazole, 3,4,5-triamino pyrazole, 1-methyl-3,4,5-triamino pyrazole, 3,5-diamino-1-methyl-4-methylamino pyrazole, 3,5-diamino-4-(β-hydroxyethyl)amino-1-methyl pyrazole, and the addition salts thereof.

The at least one additional oxidation base may be present in the composition in an amount ranging from 0.0001 to 20 wt. %, for example, from 0.005 to 6 wt. %, relative to the total weight of the composition.

Direct Dyes

The dyeing composition according to the present disclosure may also comprise at least one direct dye chosen, for example, from neutral, acidic, and cationic nitrogen-containing dyes of the benzene series, neutral, acidic, and cationic direct azo dyes, neutral, acidic, and cationic quinone, for example, anthraquinone, direct dyes, azine direct dyes, methine direct dyes, azomethine direct dyes, triarylmethane direct dyes, indoamine direct dyes, and natural direct dyes. In one embodiment, the composition according to the present disclosure may further comprise at least one dye chosen from cationic direct dyes and natural direct dyes.

Suitable cationic direct dyes may include, for example, the cationic azo direct dyes described in International Patent Application Nos. WO 95/15144 and WO 95/01772 and European Patent Application No. 0 714 954.

Non-limiting examples of these compounds include:
1,3-dimethyl-2-[[4-(dimethylamino)phenyl]azo]-1H-imidazolium chloride,
1,3-dimethyl-2-[(4-aminophenyl)azo]-1H-imidazolium chloride, and
1-methyl-4-[(methylphenylhydrazono)methyl]-pyridinium methylsulphate.

Natural direct dyes suitable for use in accordance with the present disclosure may include, for example, lawsone, juglone, alizarin, purpurin, carminic acid, kermesic acid, purpurogallin, protocatechaldehyde, indigo, isatin, curcumine, spinulosine, and apigenidine. In one embodiment, extracts or decoctions comprising these natural dyes, for instance, henna-based cataplasms or extracts, may also be used.

The at least one direct dye may be present in the composition in an amount ranging from 0.001 to 20 wt. % relative to the total weight of the composition, for example, from 0.005 to 10 wt. %.

Dyeing Medium

The medium suitable for dyeing may be chosen from water and mixtures of water and at least one organic solvent, for example, linear and branched C₁-C₄ lower alcohols, such as ethanol and isopropanol; polyols and polyol ethers, such as 2-butoxyethanol, propyleneglycol, monomethyl ether of propyleneglycol, monoethyl ether of diethyleneglycol, monomethyl ether of diethyleneglycol, and glycerol; aromatic alcohols, such as benzyl alcohol and phenoxyethanol; and mixtures thereof.

The at least one solvent may be present in the composition in an amount ranging from 1 to 40 wt. % relative to the total weight of the dyeing composition, for example, from 5 to 30 wt. %.

Cosmetic Additives

The dyeing composition of the present disclosure may also comprise at least one cosmetic additive chosen from antioxidants, penetrants, sequestering agents, perfumes, buffers, dispersants, surfactants, conditioners, film-forming agents, polymers, ceramides, preservatives, lustre agents, opacifiers, vitamins, and provitamins.

The at least one cosmetic additive may be present in the composition in an amount, for each additive, ranging from 0.01 to 20 wt. % relative to the total weight of the composition.

The pH of the composition according to the present disclosure may range from 3 to 12, for example, from 5 to 11. The pH may be adjusted to a desired value by means of acidifying or alkalizing agents conventionally employed in the dyeing of keratin fibers, or alternatively, by means of conventional buffering systems.

Examples of acidifying agents include, but are not limited to, inorganic or organic acids other than the dicarboxylic acids, such as hydrochloric acid, orthophosphoric acid, and sulphuric acid; carboxylic acids, such as acetic acid, tartaric acid, citric acid, and lactic acid; and sulphonic acids.

Suitable alkalizing agents may include, for example: ammonia, alkali metal carbonates, alkanolamines, such as mono-, di-, and triethanolamines, and derivatives thereof, sodium hydroxides, potassium hydroxides, and compounds of formula (III): 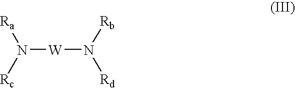
wherein:

W is chosen from propylene residues optionally substituted with an entity chosen from hydroxyl groups and C₁-C₄ alkyl radicals; and

Rₐ, R_{b}, R_{c}, and R_{d}, which may be identical or different, are chosen from hydrogen, C₁-C₄ alkyl radicals, and C₁-C₄ hydroxyalkyl radicals.

It is understood that a person skilled in the art will ensure that the envisaged optional additions, for example, the addition of at least one additive, at least one precursor of additional oxidation dyes, at least one oxidation coupler, and/or at least one direct dye, are chosen in such a way that the beneficial properties intrinsically associated with the oxidation dyeing composition according to the invention are not, or are not substantially, adversely affected.

The dyeing composition according to the present disclosure may be in various forms, such as liquids, creams, and gels, or any other form suitable for dyeing keratin fibers, such as human hair.

Method For Dyeing

Further disclosed herein is a method for dyeing keratin fibers comprising applying a dyeing composition of the present disclosure to the keratin fibers, and developing the color by means of at least one oxidizing agent. The color may be developed at acidic, neutral, or alkaline pH. In one embodiment, the at least one oxidizing agent may be added to the composition of the present disclosure at the time of use. According to another embodiment, the at least one oxidizing agent may be applied via an oxidizing composition comprising it, which may be applied simultaneously or sequentially with the composition of the present disclosure.

Non-limiting examples of oxidizing agents include hydrogen peroxide; urea peroxide; bromates of alkali metals; persalts, such as perborates and persulphates; peracids; and oxidase enzymes, for example, peroxidases, 2-electron oxido-reductases, such as uricases, and 4-electron oxygenases, such as the laccases. In one embodiment, the at least one oxidizing agent is hydrogen peroxide.

According to another embodiment, the composition according to the present disclosure may be mixed, for example, at the moment of use, with a composition comprising, in a medium suitable for dyeing, at least one oxidizing agent, this oxidizing agent being present in a sufficient amount for dye development. The mixture obtained may then be applied to the keratin fibers. After a period of coloration time ranging from 3 to 50 minutes, for instance, from 5 to 30 minutes, the keratin fibers may be rinsed, washed with shampoo, rinsed again, and then dried.

The oxidizing composition may comprise at least one of various additives used conventionally in compositions for hair dyeing and as defined above.

The pH of the oxidizing composition comprising the at least one oxidizing agent may be such that, after mixing with the dyeing composition, the pH of the resulting composition applied to the keratin fibers ranges from 3 to 12, for instance, from 5 to 11. The pH may be adjusted to a desired value via acidifying or alkalizing agents conventionally employed in the dyeing of keratin fibers and as defined above.

The ready-to-use composition that is finally applied to the keratin fibers may be in various forms, such as liquids, creams, and gels, or any other form suitable for dyeing keratin fibers, for instance, human keratin fibers, such as the hair.

The present disclosure also relates to the use of the cosmetic composition as disclosed herein, for the dyeing of keratin fibers, including human keratin fibers such as the hair.

Multi-Compartment Device

Also disclosed herein is a multi-compartment device or dyeing “kit” comprising at least one first compartment containing at least one dyeing composition comprising at least one para-phenylenediamine oxidation base chosen from compounds of formula (I) and the addition salts thereof, and at least one second compartment containing at least one oxidizing composition. This kit may be equipped with an applicator for delivering the desired mixture onto the hair, such as the kits described in French Patent No. 2 586 913.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the present disclosure are approximations, unless otherwise indicated the numerical values set forth in the specific examples are reported as precisely as possible. Any numerical value, however, inherently contain certain errors necessarily resulting from the standard deviation found in their respective testing measurements.

By way of non-limiting illustration, concrete examples of certain embodiments of the present disclosure are given below.

### EXAMPLES

### Synthesis Examples

### Example 1

### Synthesis of N-(4-aminophenyl)-N-[2-({2-[(4-aminophenyl)amino]ethyl}-amino)ethyl]amine tetrahydrochloride (2)

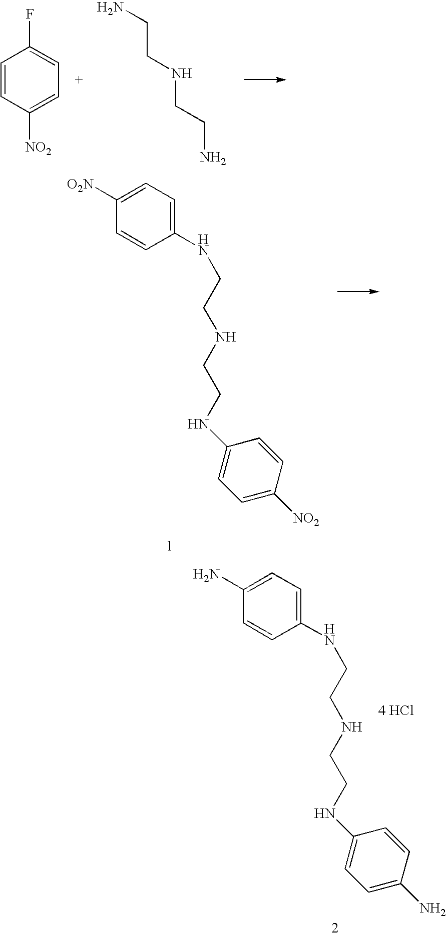

### Stage 1: Synthesis of N-(4-nitrophenyl)-N-[2-({2-[(4-nitrophenyl)amino]ethyl}-amino)ethyl]amine (1)

5 g of 4-fluoronitrobenzene (2 eq.) were dissolved in 5 ml of DMSO. 1 equivalent of diethylenetriamine and 2.1 equivalents of triethylamine were added to the solution. The reaction mixture was heated at 60° C. for 20 hours. The mixture was then poured onto crushed ice; a precipitate formed. The latter was filtered, washed with water, and then dried.

### Stage 2: Synthesis of N-(4-aminophenyl)-N-[2-({2-[(4-aminophenyl)amino]-ethyl}amino)ethyl]amine tetrahydrochloride (2)

The nitrogen-containing compound (1) above was dissolved in 500 ml of ethanol in a 1 L hydrogenator. 10% of palladium on charcoal (50% water content) was added and the hydrogenator was charged with hydrogen. After reaction for 1 h 45 min, the palladium was filtered and 20 ml of ethanol-hydrochloric acid 3M then 300 ml of isopropyl ether were added to the filtrate. The precipitate obtained was filtered and recrystallized from ethanol-hydrochloric acid.

The proton and mass NMR spectra corresponded to the expected structure of the product.

### Example 2

### Synthesis of N-(4-aminophenyl)-N-[2-({2-[(4-aminophenyl)amino]-ethyl}sulphanyl)ethyl]amine tetrahydrochloride (4)

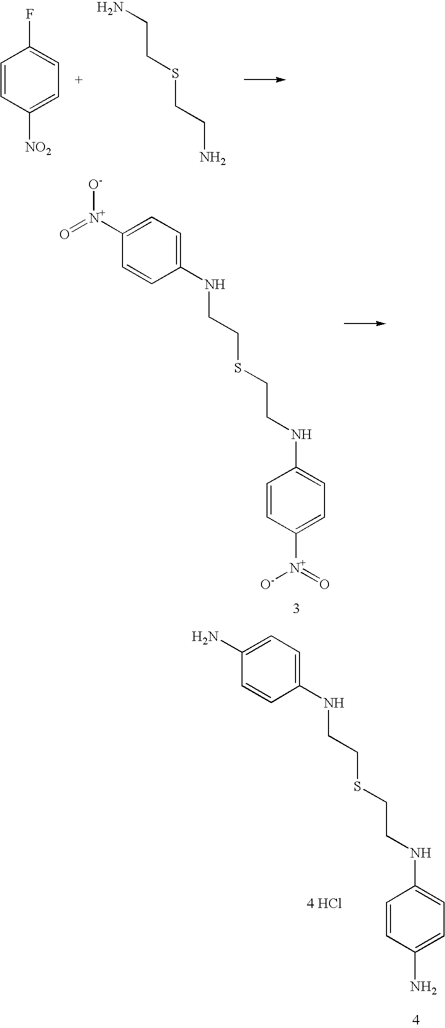

### Stage 1: Synthesis of 4-nitro-N-[2-({2-[(4-nitrophenyl)amino]ethyl}sulphanyl)ethyl]aniline (3)

2 g of 4-fluoronitrobenzene (2 eq.) were dissolved in 50 ml of DMSO. 1.2 equivalents of 2,2′-diaminodiethylsulphide and 4 equivalents of triethylamine were added to the solution. The reaction mixture was heated at 80° C. for 20 hours. The mixture was then poured onto crushed ice; a precipitate formed. The latter was filtered, washed with water, and then dried.

### Stage 2: Synthesis of N-(4-aminophenyl)-N-[2-({2-[(4-aminophenyl)amino]ethyl}-sulphanyl)ethyl]amine tetrahydrochloride (4)

The nitrogen-containing compound (3) was dissolved in 500 ml of ethanol in a 1 L hydrogenator. 10% of palladium on charcoal (50% water content) was added and the hydrogenator was charged with hydrogen. After reaction for 1 h 30 min, the palladium was filtered and 20 ml of ethanol-hydrochloric acid 3M then 300 ml of isopropyl ether were added to the filtrate. The precipitate obtained was filtered and recrystallized from ethanol-hydrochloric acid.

The proton and mass NMR spectra corresponded to the expected structure of the product.

### Example 3

### Synthesis of N-(4-aminophenyl)-N-{2-[{2-[(4-aminophenyl)-amino]ethyl}(methyl)amino]ethyl}amine tetrahydrochloride (6)

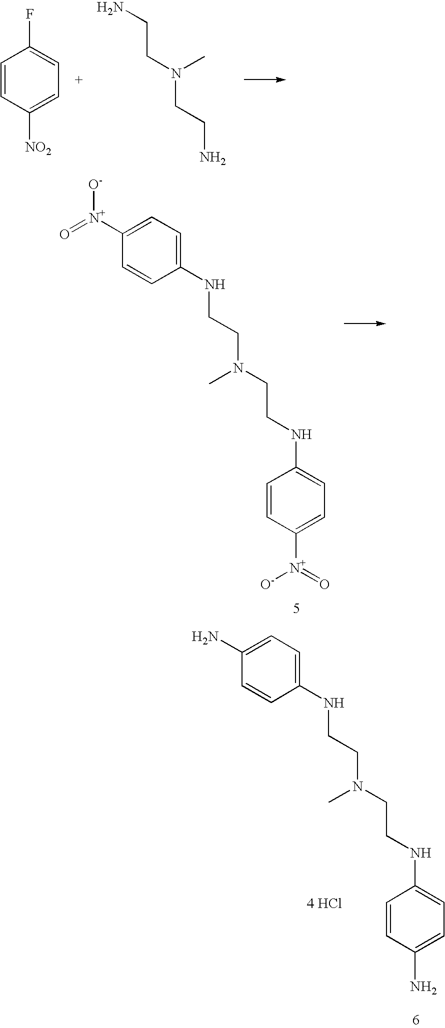

### Stage 1: Synthesis of N-methyl-N,N-bis{2-[(4-nitrophenyl)amino]ethyl}amine (5)

2 g of 5-fluoro-2-nitrotoluene (2 eq.) were dissolved in 5 ml of DMSO. 1.2 equivalents of N-methyl-2,2′-diaminodiethylamine and 4 equivalents of triethylamine were added to the solution. The reaction mixture was heated at 60° C. for 7 hours. The mixture was then poured onto crushed ice; a precipitate formed. The latter was filtered, washed with water, and then dried.

### Stage 2: Synthesis of N-(4-aminophenyl)-N-{2-[{2-[(4-aminophenyl)amino]ethyl}-(methyl)amino]ethyl}amine tetrahydrochloride (6)

The nitrogen-containing compound (5) above was dissolved in 500 ml of ethanol in a 1 L hydrogenator. 10% of palladium on charcoal (50% water content) was added and the hydrogenator was charged with hydrogen. After reaction for 1 h 30 min, the palladium was filtered and 20 ml of ethanol-hydrochloric acid 3M then 300 ml of isopropyl ether were added to the filtrate. The precipitate obtained was filtered and recrystallized from ethanol-hydrochloric acid.

The proton and mass NMR spectra corresponded to the expected structure of the product.

### Example 4

### Synthesis of N-methyl-N,N-bis {2-[(2-methyl-4-aminophenyl)amino]ethyl}amine tetrahydrochloride (8)

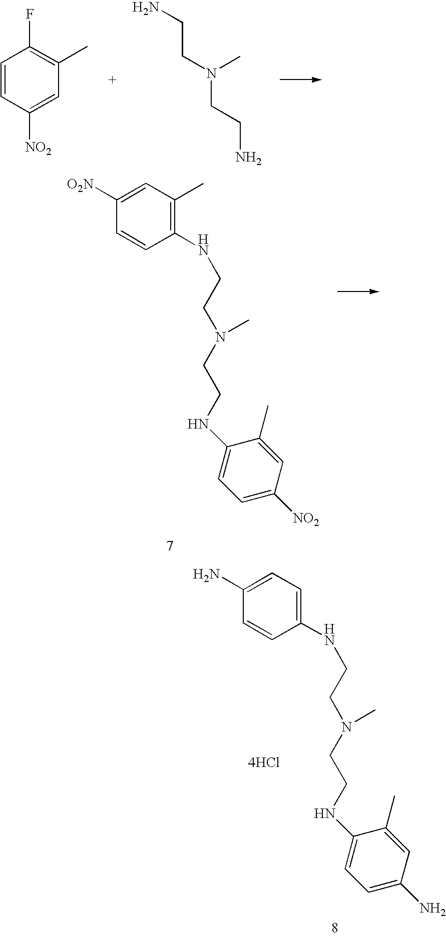

### Stage 1: Synthesis of N-methyl-N,N-bis{2-[(2-methyl-4-nitrophenyl)amino]ethyl}amine (7)

1.5 g of 2-fluoro-5-nitrotoluene (2eq.) were dissolved in 10 ml of DMSO. 1.2 equivalents of N-(2-aminoethyl)-N-methylethane-1,2-diamine and 1.2 equivalents of triethylamine were added to the solution. The reaction mixture was heated at 80° C. for 24 hours. The mixture was then poured onto crushed ice; a precipitate formed. The latter was filtered, washed with water, and then dried.

### Stage 2: Synthesis of N-methyl-N,N-bis{2-[(2-methyl-4-aminophenyl)amino]ethyl}amine tetrahydrochloride (8)

The product obtained in the preceding stage was reduced with a boiling zinc/ammonium chloride/water/ethanol mixture. The corresponding amine was isolated in the form of the tetrahydrochloride.

The proton and mass NMR spectra corresponded to the expected structure of the product.

### Example 5

### Synthesis of N-methyl-N,N-bis{2-[(3-methyl-4-aminophenyl)-amino]ethyl}amine tetrahydrochloride (10)

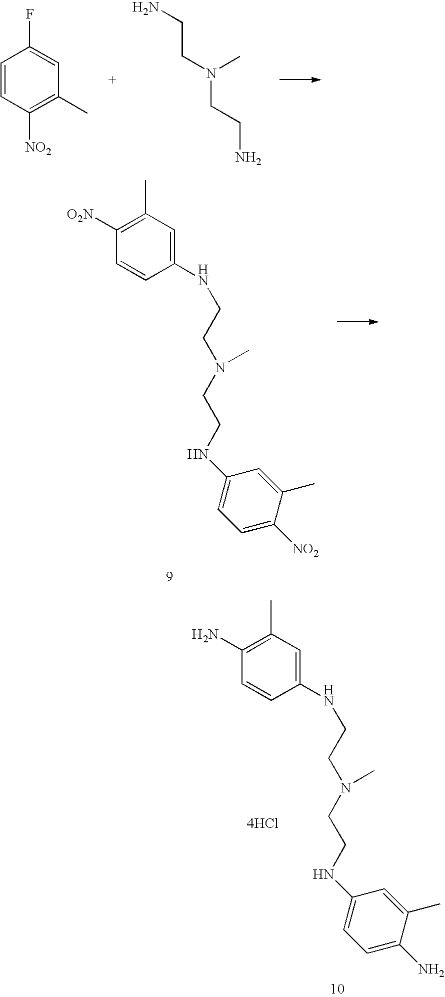

### Stage 1: Synthesis of N-methyl-N,N-bis{2-[(3-methyl-4-nitrophenyl)amino]ethyl}amine (8)

1.5 g of 5-fluoro-2-nitrotoluene (2 eq.) were dissolved in 10 ml of DMSO. 1.2 equivalents of N-(2-aminoethyl)-N-methylethane-1,2-diamine and 1.2 equivalents of triethylamine were added to the solution. The reaction mixture was heated at 80° C. for 24 hours. The mixture was then poured onto crushed ice; a precipitate formed. The latter was filtered, washed with water, and then dried.

### Stage 2: Synthesis of N-methyl-N,N-bis{2-[(3-methyl-4-aminophenyl)amino]ethyl}amine (10)

The product (8) obtained in the preceding stage was reduced with a boiling zinc/ammonium chloride/water/ethanol mixture. The corresponding amine was isolated in the form of the tetrahydrochloride.

The proton and mass NMR spectra corresponded to the expected structure of the product.

Examples of Dyeing

### Examples 1 to 12

### Dyeing composition based on N-(4-aminophenyl)-N-[2-({2-[(4-aminophenyl)amino]ethyl}amino)ethyl]amine tetrahydrochloride (2)

### Examples 1 to 6

### Dyeing in an Acid Medium

The following dyeing compositions were prepared:

| | Example | | | | | |
|---|---|---|---|---|---|---|
| | 1 | 2 | 3 | 4 | 5 | 6 |
| N-(4-aminophenyl)-N-[2-({2-[(4- | 10⁻³ mol | 10⁻³ mol | 10⁻³ mol | 10⁻³ mol | 10⁻³ mol | 10⁻³ mol |
| aminophenyl)- | | | | | | |
| amino]ethyl}amino)ethyl]amine | | | | | | |
| tetrahydrochloride (2) | | | | | | |
| Benzene-1,3-diol | 10⁻³ mol | | | | | |
| 5-Amino-2-methyl-phenol | | 10⁻³ mol | | | | |
| 1H-Indol-6-ol | | | 10⁻³ mol | | | |
| 2-Amino-pyridin−3-ol | | | | 10⁻³ mol | | |
| 3,6-Dimethyl-1H-pyrazolo | | | | | 10⁻³ mol | |
| [5,1-c][1,2,4]triazole | | | | | | |
| 2-(2,4-Diamino-phenoxy)-ethanol | | | | | | 10⁻³ mol |
| hydrochloride | | | | | | |
| Dyeing support (2) | (*) | (*) | (*) | (*) | (*) | (*) |
| Demineralized water q.s.f. | 100 g | 100 g | 100 g | 100 g | 100 g | 100 g |

(*): dyeing support (1) pH 7

| | | |
|---|---|---|
| 96° ethanol | 20.8 | g |
| Sodium metabisulphite, 35% aqueous solution | 0.23 | g M.A |
| Pentasodium salt of diethylene-triamine-pentaacetic | 0.48 | g M.A |
| acid, 40% aqueous solution | | |
| C₈-C₁₀ alkyl polyglycoside, 60% aqueous solution | 3.6 | g M.A |
| Benzyl alcohol | 2.0 | g |
| Polyethylene glycol with 8 ethylene oxide units | 3.0 | g |
| Na₂HPO₄ | 0.28 | g |
| KH₂PO₄ | 0.46 | g |

At the time of use, each composition was mixed with an equal weight of hydrogen peroxide at 20 volumes (6 wt. %). A final pH of 7 was obtained.

Each mixture obtained was applied to locks of grey hair comprising 90% white hairs. After a period of coloration time of 30 minutes, the locks were rinsed, washed with a standard shampoo, rinsed again, and then dried.

The shades obtained are shown in the following table:

| | Example | | | | | |
|---|---|---|---|---|---|---|
| | 1 | 2 | 3 | 4 | 5 | 6 |
| Shade observed | deep | deep | deep | deep | deep | deep grey |
| | grey | grey | brown | grey | brown | violet-blue |

### Examples 7 to 12

### Dyeing in a Basic Medium

The following dyeing compositions were prepared:

| | Example | | | | | |
|---|---|---|---|---|---|---|
| | 7 | 8 | 9 | 10 | 11 | 12 |
| N-(4-aminophenyl)-N-[2- | 10⁻³ mol | 10⁻³ mol | 10⁻³ mol | 10⁻³ mol | 10⁻³ mol | 10⁻³ mol |
| ({2-[(4- | | | | | | |
| aminophenyl)amino]ethyl}- | | | | | | |
| amino)ethyl]amine | | | | | | |
| tetrahydrochloride (2) | | | | | | |
| Benzene-1,3-diol | 10⁻³ mol | | | | | |
| 5-Amino-2-methyl-phenol | | 10⁻³ mol | | | | |
| 1H-Indol-6-ol | | | 10⁻³ mol | | | |
| 2-Amino-pyridin−3-ol | | | | 10⁻³ mol | | |
| 3,6-Dimethyl-1H-pyrazolo | | | | | 10⁻³ mol | |
| [5,1-c][1,2,4]triazole | | | | | | |
| 2-(2,4-Diamino-phenoxy)- | | | | | | 10⁻³ mol |
| ethanol hydrochloride | | | | | | |
| Dyeing support (2) | (*) | (*) | (*) | (*) | (*) | (*) |
| Demineralized water q.s.f. | 100 g | 100 g | 100 g | 100 g | 100 g | 100 g |

(*): dyeing support (2) pH 9.5

| | | |
|---|---|---|
| 96° ethanol | 20.8 | g |
| Sodium metabisulphite, 35% aqueous solution | 0.23 | g M.A |
| Pentasodium salt of diethylene-triamine-pentaacetic | 0.48 | g MA |
| acid, 40% aqueous solution | | |
| C₈-C₁₀ alkyl polyglycoside, 60% aqueous solution | 3.6 | g M.A |
| Benzyl alcohol | 2.0 | g |
| Polyethylene glycol with 8 ethylene oxide units | 3.0 | g |
| NH₄Cl | 4.32 | g |
| Ammonia at 20% NH₃ | 2.94 | g |

At the time of use, each composition was mixed with an equal weight of hydrogen peroxide at 20 volumes (6 wt. %). A final pH of 9.5 was obtained.

Each mixture obtained was applied to locks of grey hair comprising 90% white hairs. After a period of coloration time of 30 minutes, the locks were rinsed, washed with a standard shampoo, rinsed again, and then dried.

The shades obtained are shown in the following table:

| | Example | | | | | |
|---|---|---|---|---|---|---|
| | 7 | 8 | 9 | 10 | 11 | 12 |
| Shade | orangey | deep | deep | deep | deep | deep |
| observed | brown | red- | orangey | orangey | red | grey |
| | | brown | brown | brown | | blue |

### Examples 13 to 26

### Dyeing composition based on N-(4-aminophenyl)-N-[2-({2-[(4-aminophenyl)amino]ethyl}sulphanyl)ethyl]amine tetrahydrochloride (4)

### Examples 13 to 19

### Dyeing in an Acid Medium

The following dyeing compositions were prepared:

| | Example | | | | | | |
|---|---|---|---|---|---|---|---|
| | 13 | 14 | 15 | 16 | 17 | 18 | 19 |
| N-(4-aminophenyl)-N-[2-({2-[(4-amino- | 10⁻³ mol | 10⁻³ mol | 10⁻³ mol | 10⁻³ mol | 10⁻³ mol | 10⁻³ mol | 10⁻³ mol |
| phenyl)amino]ethyl}sulphanyl)ethyl]amine | | | | | | | |
| tetrahydrochloride (4) | | | | | | | |
| Benzene-1,3-diol | 10⁻³ mol | | | | | | |
| 5-Amino-2-methyl-phenol | | 10⁻³ mol | | | | | |
| 1H-Indol-6-ol | | | 10⁻³ mol | | | | |
| 2-Amino-pyridin−3-ol | | | | 10⁻³ mol | | | |
| 3,6-Dimethy-1H-pyrazolo | | | | | 10⁻³ mol | | |
| [5,1-c][1,2,4]-triazole | | | | | | | |
| 2-(2,4-Diamino-phenoxy)-ethanol | | | | | | 10⁻³ mol | |
| hydrochloride | | | | | | | |
| 3-Amino-2-chloro-6-methyl-phenol | | | | | | | 10⁻³ mol |
| hydrochloride | | | | | | | |
| Dyeing support (1) | (*) | (*) | (*) | (*) | (*) | (*) | (*) |
| Demineralized water q.s.f. | 100 g | 100 g | 100 g | 100 g | 100 g | 100 g | 100 g |

(*): dyeing support (1) as described above

At the time of use, each composition was mixed with an equal weight of hydrogen peroxide at 20 volumes (6 wt. %). A final pH of 7 was obtained.

Each mixture obtained was applied to locks of grey hair comprising 90% white hairs. After a period of coloration time of 30 minutes, the locks were rinsed, washed with a standard shampoo, rinsed again, and then dried.

The shades obtained are shown in the following table:

| | Example | | | | | | |
|---|---|---|---|---|---|---|---|
| | 13 | 14 | 15 | 16 | 17 | 18 | 19 |
| Shade | orangey | deep | deep | deep | red | deep | deep |
| observed | brown | red | brown | brown | | violet- | violet |
| | | | red | red | | blue | |

### Examples 20 to 26

### Dyeing in a Basic Medium

The following dyeing compositions were prepared:

| | Example | | | | | | |
|---|---|---|---|---|---|---|---|
| | 20 | 21 | 22 | 23 | 24 | 25 | 26 |
| N-(4-aminophenyl)-N-[2-({2-[(4-amino- | 10⁻³ mol | 10⁻³ mol | 10⁻³ mol | 10⁻³ mol | 10⁻³ mol | 10⁻³ mol | 10⁻³ mol |
| phenyl)amino]ethyl}sulphanyl)ethyl]amine | | | | | | | |
| tetrahydrochloride (4) | | | | | | | |
| Benzene-1,3-diol | 10⁻³ mol | | | | | | |
| 5-Amino-2-methyl-phenol | | 10⁻³ mol | | | | | |
| 1H-Indol-6-ol | | | 10⁻³ mol | | | | |
| 2-Amino-pyridin−3-ol | | | | 10⁻³ mol | | | |
| 3,6-Dimethyl-1H-pyrazolo | | | | | 10⁻³ mol | | |
| [5,1-c][1,2,4]triazole | | | | | | | |
| 2-(2,4-Diamino-phenoxy)-ethanol | | | | | | 10⁻³ mol | |
| hydrochloride | | | | | | | |
| 3-Amino-2-chloro-6-methyl-phenol | | | | | | | 10⁻³ mol |
| hydrochloride | | | | | | | |
| Dyeing support (2) | (*) | (*) | (*) | (*) | (*) | (*) | (*) |
| Demineralized water q.s.f. | 100 g | 100 g | 100 g | 100 g | 100 g | 100 g | 100 g |

(*): dyeing support (2) as defined previously

At the time of use, each composition was mixed with an equal weight of hydrogen peroxide at 20 volumes (6 wt. %). A final pH of 9.5 was obtained.

Each mixture obtained was applied to locks of grey hair comprising 90% white hairs. After a period of coloration time of 30 minutes, the locks were rinsed, washed with a standard shampoo, rinsed again, and then dried.

The shades obtained are shown in the following table:

| | Example | | | | | | |
|---|---|---|---|---|---|---|---|
| | 20 | 21 | 22 | 23 | 24 | 25 | 26 |
| Shade | orangey | deep | orangey | orangey | red | deep | grey |
| observed | | red | | | | violet- | violet- |
| | | | | | | blue | red |

### Examples 27 to 40

### Dyeing composition based on N-(4-aminophenyl)-N-{2-[{2-[(4-aminophenyl)amino]ethyl}(methyl)amino]ethyl}amine (6)

### Examples 27 to 33

### Dyeinq in an Acidic Medium

The following dyeing compositions were prepared:

| | Example | | | | | | |
|---|---|---|---|---|---|---|---|
| | 27 | 28 | 29 | 30 | 31 | 32 | 33 |
| N-(4-aminophenyl)-N-{2-[{2-[(4-amino- | 10⁻³ mol | 10⁻³ mol | 10⁻³ mol | 10⁻³ mol | 10⁻³ mol | 10⁻³ mol | 10⁻³ mol |
| phenyl)amino]ethyl}(methyl)amino]ethyl} | | | | | | | |
| amine tetrahydrochloride (6) | | | | | | | |
| Benzene-1,3-diol | 10⁻³ mol | | | | | | |
| 5-Amino-2-methyl-phenol | | 10⁻³ mol | | | | | |
| 1H-Indol-6-ol | | | 10⁻³ mol | | | | |
| 2-Amino-pyridin−3-ol | | | | 10⁻³ mol | | | |
| 3,6-Dimethyl-1H-pyrazolo | | | | | 10⁻³ mol | | |
| [5,1-c][1,2,4]triazole | | | | | | | |
| 2-(2,4-Diaminophenoxy)-ethanol | | | | | | 10⁻³ mol | |
| hydrochloride | | | | | | | |
| 3-Amino-2-chloro-6-methyl-phenol | | | | | | | 10⁻³ mol |
| hydrochloride | | | | | | | |
| Dyeing support (1) | (*) | (*) | (*) | (*) | (*) | (*) | (*) |
| Demineralized water q.s.f. | 100 g | 100 g | 100 g | 100 g | 100 g | 100 g | 100 g |

(*): dyeing support (1) as defined above

At the time of use, each composition was mixed with an equal weight of hydrogen peroxide at 20 volumes (6 wt. %). A final pH of 7 was obtained.

Each mixture obtained was applied to locks of grey hair comprising 90% white hairs. After a period of coloration time of 30 minutes, the locks were rinsed, washed with a standard shampoo, rinsed again, and then dried.

The shades obtained are shown in the following table:

| | Example | | | | | | |
|---|---|---|---|---|---|---|---|
| | 27 | 28 | 29 | 30 | 31 | 32 | 33 |
| Shade | deep | deep | deep | deep | deep | deep | deep |
| observed | orangey | red- | red- | red- | red- | grey | grey |
| | brown | brown | brown | brown | brown | violet- | violet- |
| | | | | | | blue | red |

### Examples 34 to 40

### Dyeing in a Basic Medium

The following dyeing compositions were prepared:

| | Example | | | | | | |
|---|---|---|---|---|---|---|---|
| | 34 | 35 | 36 | 37 | 38 | 39 | 40 |
| N-(4-aminophenyl)-N-{2-[{2-[(4-amino- | 10⁻³ mol | 10⁻³ mol | 10⁻³ mol | 10⁻³ mol | 10⁻³ mol | 10⁻³ mol | 10⁻³ mol |
| phenyl)amino]ethyl}(methyl)amino]ethyl} | | | | | | | |
| amine tetrahydrochloride (6) | | | | | | | |
| Benzene-1,3-diol | 10⁻³ mol | | | | | | |
| 5-Amino-2-methyl-phenol | | 10⁻³ mol | | | | | |
| 1H-Indol-6-ol | | | 10⁻³ mol | | | | |
| 2-Amino-pyridin-3-ol | | | | 10⁻³ mol | | | |
| 3,6-Dimethyl-1H-pyrazolo | | | | | 10⁻³ mol | | |
| [5,1-c][1,2,4]triazole | | | | | | | |
| 2-(2,4-Diaminophenoxy)-ethanol | | | | | | 10⁻³ mol | |
| hydrochloride | | | | | | | |
| 3-Amino-2-chloro-6-methyl-phenol | | | | | | | 10⁻³ mol |
| hydrochloride | | | | | | | |
| Dyeing support (2) | (*) | (*) | (*) | (*) | (*) | (*) | (*) |
| Demineralized water q.s.f. | 100 g | 100 g | 100 g | 100 g | 100 g | 100 g | 100 g |

(*): dyeing support (2) as defined above

At the time of use, each composition was mixed with an equal weight of hydrogen peroxide at 20 volumes (6 wt. %). A final pH of 9.5 was obtained.

Each mixture obtained was applied to locks of grey hair comprising 90% white hairs. After a period of coloration time of 30 minutes, the locks were rinsed, washed with a standard shampoo, rinsed again, and then dried.

The shades obtained are shown in the following table:

| | Example | | | | | | |
|---|---|---|---|---|---|---|---|
| | 34 | 35 | 36 | 37 | 38 | 39 | 40 |
| Shade | deep | deep | deep | deep | deep | deep grey | deep |
| observed | orangey | red | orangey | red | red | violet-blue | violet |

### Examples 41 to 50

### Dyeing composition based on N-methyl-N,N-bis{2-[(2-methyl-4-aminophenyl)amino]ethyl}amine (8)

### Examples 41 to 45

### Dyeing in an Acidic Medium

The following dyeing compositions were prepared:

| | Example | | | | |
|---|---|---|---|---|---|
| | 41 | 42 | 43 | 44 | 45 |
| N-methyl-N,N-bis{2-[(2-methyl-4- | 10⁻³ mol | 10⁻³ mol | 10⁻³ mol | 10⁻³ mol | 10⁻³ mol |
| aminophenyl)amino]ethyl}amine | | | | | |
| tetrahydrochloride (8) | | | | | |
| 5-Amino-2-methyl-phenol | 10⁻³ mol | | | | |
| 1H-Indol-6-ol | | 10⁻³ mol | | | |
| 2-Amino-pyridin-3-ol | | | 10⁻³ mol | | |
| 2-(2,4-Diamino-phenoxy)-ethanol | | | | 10⁻³ mol | |
| hydrochloride | | | | | |
| 3-Amino-2-chloro-6-methyl-phenol | | | | | 10⁻³ mol |
| hydrochloride | | | | | |
| Dyeing support (1) | (*) | (*) | (*) | (*) | (*) |
| Demineralized water q.s.f. | 100 g | 100 g | 100 g | 100 g | 100 g |

(*): dyeing support (1) as defined above

At the time of use, each composition was mixed with an equal weight of hydrogen peroxide at 20 volumes (6 wt. %). A final pH of 7 was obtained.

Each mixture obtained was applied to locks of grey hair at 90% white. After a period of coloration time of 30 minutes, the locks were rinsed, washed with a standard shampoo, rinsed again, and then dried.

The shades obtained are given in the following table:

Example

| | 41 | 42 | 43 | 44 | 45 |
|---|---|---|---|---|---|
| Shade | red | deep grey | orangey | deep | deep |
| observed | | violet | | violet- | violet- |
| | | | | blue | red |

### Examples 46 to 50

### Dyeing in a Basic Medium

The following dyeing compositions were prepared:

| | Example | | | |
|---|---|---|---|---|
| | 46 | 47 | 48 | 49 |
| N-methyl-N,N- | 10⁻³ mol | 10⁻³ mol | 10⁻³ mol | 10⁻³ mol |
| bis{2-[(2-methyl- | | | | |
| 4-aminophenyl)amino- | | | | |
| ]ethyl}amine | | | | |
| tetrahydrochloride (8) | | | | |
| 5-Amino-2-methyl- | 10⁻³ mol | | | |
| phenol | | | | |
| 1H-Indol-6-ol | | 10⁻³ mol | | |
| 2-(2,4-Diamino- | | | 10⁻³ mol | |
| phenoxy)-ethanol | | | | |
| hydrochloride | | | | |
| 3-Amino-2-chloro- | | | | 10⁻³ mol |
| 6-methyl-phenol | | | | |
| hydrochloride | | | | |
| Dyeing support (2) | (*) | (*) | (*) | (*) |
| Demineralized | 100 g | 100 g | 100 g | 100 g |
| water q.s.f. | | | | |

(*) dyeing support (2) as defined above

At the time of use, each composition was mixed with an equal weight of hydrogen peroxide at 20 volumes (6 wt. %). A final pH of 9.5 was obtained.

Each mixture obtained was applied to locks of grey hair comprising 90% white hairs. After a period of coloration time of 30 minutes, the locks were rinsed, washed with a standard shampoo, rinsed again, and then dried.

The shades obtained are shown in the following table:

Example

| | 46 | 47 | 48 | 49 |
|---|---|---|---|---|
| Shade observed | red | orangey | deep violet- | violet |
| | | | blue | |

### Examples 50 to 58

### Dyeing composition based on N-methyl-N,N-bis{2-[(3-methyl-4-aminophenyl)amino]ethyl}amine tetrahydrochloride (10)

### Examples 50 to 54

### Dyeing in an Acidic Medium

The following dyeing compositions were prepared:

| | Example | | | | |
|---|---|---|---|---|---|
| | 50 | 51 | 52 | 53 | 54 |
| N-methyl-N,N-bis{2-[(3-methyl-4- | 10⁻³ mol | 10⁻³ mol | 10⁻³ mol | 10⁻³ mol | 10⁻³ mol |
| aminophenyl)amino]ethyl}amine | | | | | |
| tetrahydrochloride (10) | | | | | |
| 5-Amino-2-methyl-phenol | 10⁻³ mol | | | | |
| 1H-Indol-6-ol | | 10⁻³ mol | | | |
| 2-Amino-pyridin-3-ol | | | 10⁻³ mol | | |
| 2-(2,4-Diamino-phenoxy)-ethanol | | | | 10⁻³ mol | |
| hydrochloride | | | | | |
| 3-Amino-2-chloro-6-methyl-phenol | | | | | 10⁻³ mol |
| hydrochloride | | | | | |
| Dyeing support (1) | (*) | (*) | (*) | (*) | (*) |
| Demineralized water q.s.f. | 100 g | 100 g | 100 g | 100 g | 100 g |

(*) dyeing support (1) as defined above

At the time of use, each composition was mixed with an equal weight of hydrogen peroxide at 20 volumes (6 wt. %). A final pH of 7 was obtained.

Each mixture obtained was applied to locks of grey hair comprising 90% white hairs. After a period of coloration time of 30 minutes, the locks were rinsed, washed with a standard shampoo, rinsed again, and then dried.

The shades obtained are shown in the following table:

| | Example | | | | |
|---|---|---|---|---|---|
| | 50 | 51 | 52 | 53 | 54 |
| Shade | red | deep grey | orangey | deep blue | deep violet |
| observed | | | | | |

### Examples 55 to 58

### Dyeing in a Basic Medium

The following dyeing compositions were prepared:

| | Example | | | |
|---|---|---|---|---|
| | 55 | 56 | 57 | 58 |
| N-methyl-N,N- | 10⁻³ mol | 10⁻³ mol | 10⁻³ mol | 10⁻³ mol |
| bis{2-[(3-methyl- | | | | |
| 4-aminophenyl)amino- | | | | |
| ]ethyl}amine | | | | |
| tetrahydrochloride | | | | |
| (10) | | | | |
| 5-Amino-2-methyl- | 10⁻³ mol | | | |
| phenol | | | | |
| 1H-Indol-6-ol | | 10⁻³ mol | | |
| 2-(2,4-Diamino- | | | 10⁻³ mol | |
| phenoxy)-ethanol | | | | |
| hydrochloride | | | | |
| 3-Amino-2-chloro- | | | | 10⁻³ mol |
| 6-methyl-phenol | | | | |
| hydrochloride | | | | |
| Dyeing support (2) | (*) | (*) | (*) | (*) |
| Demineralized | 100 g | 100 g | 100 g | 100 g |
| water q.s.f. | | | | |

(*): dyeing support (2) as defined above

At the time of use, each composition was mixed with an equal weight of hydrogen peroxide at 20 volumes (6 wt. %). A final pH of 9.5 is obtained.

Each mixture obtained was applied to locks of grey hair comprising 90% white hairs. After a period of coloration time of 30 minutes, the locks were rinsed, washed with a standard shampoo, rinsed again, and then dried.

The shades obtained are shown in the following table:

Example

| | 55 | 56 | 57 | 58 |
|---|---|---|---|---|
| Shade observed | red | orangey | grey blue | deep violet |

What is claimed is:

1. A para-phenylenediamine compound chosen from compounds of formula (I) and the addition salts thereof: 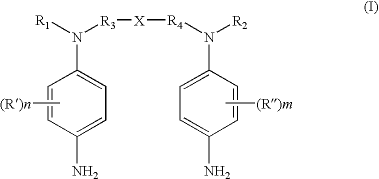
wherein:
X is chosen from:
sulphur, and
—NR₅— radicals,
R₃ and R₄, which may be identical or different, are chosen from C₂-C₆ alkylene radicals,
R₁ and R₂, which may be identical or different, are chosen from:
hydrogen,
linear and branched C₁-C₆ alkyl radicals, and
linear and branched C₁-C₆ alkyl radicals substituted by at least one radical chosen from hydroxy, C₁-C₄ alkoxy, amino, C₁-C₄ monoalkylamino, and C₁-C₄ dialkylamino radicals,
R′ and R″, which may be identical or different, are chosen from:
C₁-C₆ alkyl radicals,
C₁-C₆ alkoxy radicals,
C₁-C₆ hydroxy-alkoxy radicals,
C₁-C₆ alkoxy(C₁-C₆)alkyl radicals, and
C₁-C₆ mono- or poly-hydroxy alkyl radicals,
R₅ is chosen from:
hydrogen,
linear and branched C₁-C₁₂ alkyl radicals,
tosyl radicals,
mesyl radicals, and
benzene rings optionally functionalized with at least one group chosen from C₁-C₄ alkyl groups and 3-carboxypropyl groups, and
n and m, which may be identical or different, are chosen from integers ranging from 0 to 4.

2. The para-phenylenediamine compound of claim 1, wherein X is NR₅ and R₅ is chosen from hydrogen and C₁-C₄ alkyl radicals.

3. The para-phenylenediamine compound of claim 1, wherein R₁ and R₂, which may be identical or different, are chosen from hydrogen and optionally substituted C₁-C₄ alkyl groups.

4. The para-phenylenediamine compound of claim 1, wherein R₃ and R₄, which may be identical or different, are chosen from C₂-C₃ alkylene radicals.

5. The para-phenylenediamine compound of claim 1, wherein n and m, which may be identical or different are integers ranging from 0 to 1.

6. The para-phenylenediamine compound of claim 1, wherein the addition salts are acid addition salts chosen from hydrochloric acid, hydrobromic acid, sulphuric acid, citric acid, succinic acid, tartaric acid, lactic acid, para-toluene-sulphonic acid, benzene-sulphonic acid, phosphoric acid, and acetic acid addition salts, these compounds optionally being in the form of solvates.

7. The para-phenylenediamine compound of claim 1, chosen from the following:

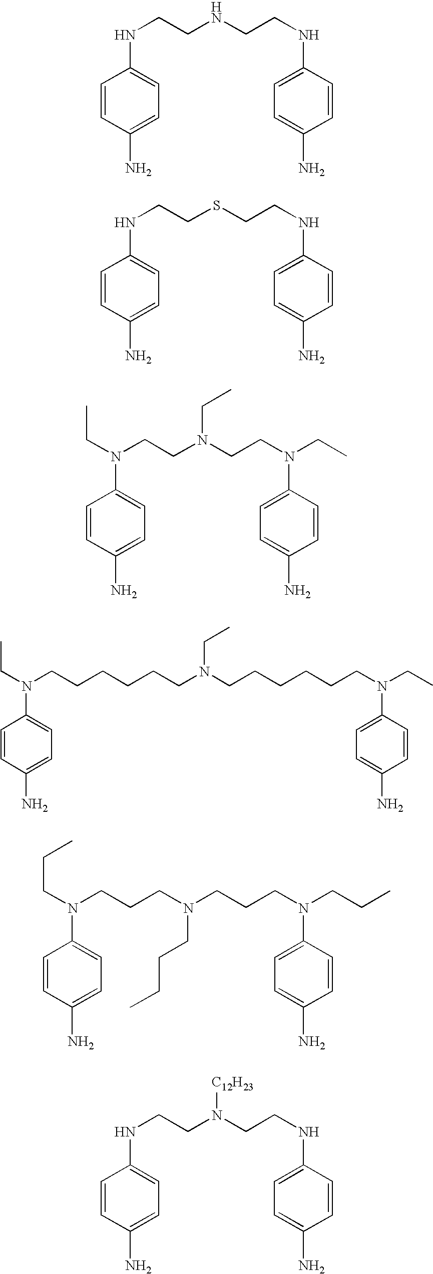

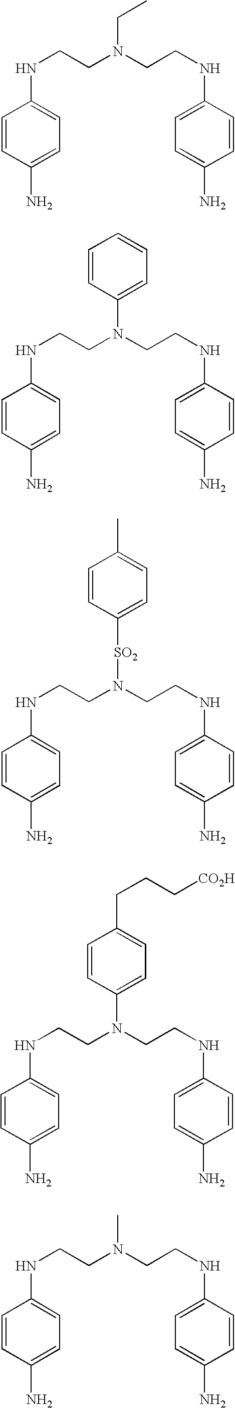

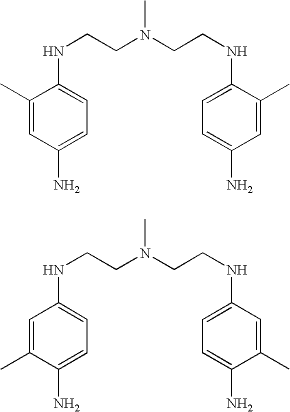

8. The para-phenylenediamine compound of claim 7, chosen from the following:

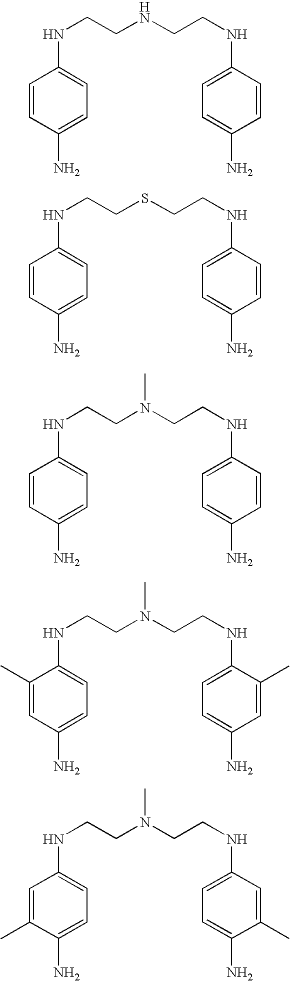

9. A dyeing composition comprising, in a medium that is suitable for dyeing, at least one para-phenylenediamine oxidation base chosen from compounds of formula (I) and the addition salts thereof: 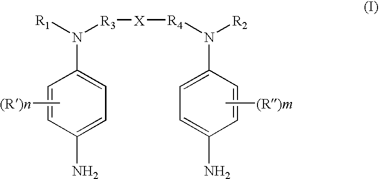
wherein:
X is chosen from:
sulphur, and
NR₅— radicals,
R₃ and R₄, which may be identical or different, are chosen from C₂-C₆ alkylene radicals,
R₁ and R₂, which may be identical or different, are chosen from:
hydrogen,
linear and branched C₁-C₆ alkyl radicals, and
linear and branched C₁-C₆ alkyl radicals substituted by at least one radical chosen from hydroxy, C₁-C₄ alkoxy, amino, C₁-C₄ monoalkylamino, and C₁-C₄ dialkylamino radicals,
R′ and R″, which may be identical or different, are chosen from:
C₁-C₆ alkyl radicals,
C₁-C₆ alkoxy radicals,
C₁-C₆ hydroxy-alkoxy radicals,
C₁-C₆ alkoxy(C₁-C₆)alkyl radicals, and
C₁-C₆ mono- and poly-hydroxy alkyl radicals,
R₅ is chosen from:
hydrogen,
linear and branched C₁-C₁₂ alkyl radicals,
tosyl radicals,
mesyl radicals, and
benzene rings optionally functionalized with at least one group chosen from C₁-C₄ alkyl groups and 3-carboxypropyl groups, and
n and m, which may be identical or different, are chosen from integers ranging from 0 to 4.

10. The composition of claim 9, wherein the at least one para-phenylenediamine oxidation base is present in the composition in an amount ranging from 0.0001 wt. % to 20 wt. % relative to the total weight of the composition

11. The composition of claim 10, wherein the at least one para-phenylenediamine oxidation base is present in the composition in an amount ranging from 0.01 wt. % to 10 wt. % relative to the total weight of the composition.

12. The composition of claim 9, further comprising at least one oxidation coupler.

13. The composition of claim 12, wherein the at least one oxidation coupler is chosen from meta-phenylenediamines, meta-aminophenols, meta-diphenols, naphthalenic couplers, heterocyclic couplers, and the addition salts thereof.

14. The composition of claim 9, further comprising at least one additional oxidation base different from the at least one oxidation base of formula (I).

15. The composition of claim 14, wherein the at least one additional oxidation base is chosen from para-phenylenediamines, bis-phenylalkylenediamines, para-aminophenols, ortho-aminophenols, heterocyclic bases, and the addition salts thereof.

16. The composition of claim 9, further comprising at least one direct dye.

17. The composition of claim 9, wherein the medium suitable for dyeing is chosen from water and mixtures of water and at least one organic solvent.

18. The composition of claim 17, wherein the at least one organic solvent is chosen from linear and branched C₁-C₄ lower alcohols, and aromatic alcohols.

19. The composition of claim 9, further comprising at least one cosmetic additive chosen from antioxidants, penetrants, sequestering agents, perfumes, buffers, dispersants, surfactants, conditioners, film-forming agents, polymers, ceramides, preservatives, lustre agents, opacifiers, vitamins, and provitamins.

20. The composition of claim 19, wherein the at least one cosmetic additive is present in the composition in an amount, for each additive, ranging from 0.01 wt. % to 20 wt. % relative to the total weight of the composition.

21. The composition of claim 9, further comprising at least one oxidizing agent.

22. A method for dyeing keratin fibers, comprising applying at least one dyeing composition to the keratin fibers in the presence of at least one oxidizing agent for a period of time sufficient to develop a desired coloration
wherein the at least one dyeing composition comprises, in a medium that is suitable for dyeing, at least one para-phenylenediamine oxidation base chosen from compounds of formula (I) and the addition salts thereof: 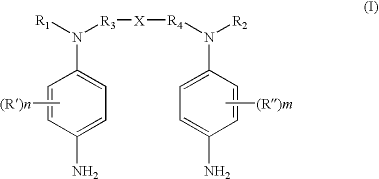
wherein:
X is chosen from:
sulphur, and
—NR₅— radicals,
R₃ and R₄, which may be identical or different, are chosen from C₂-C₆ alkylene radicals,
R₁ and R₂, which may be identical or different, are chosen from:
hydrogen,
linear and branched C₁-C₆ alkyl radicals, and
linear and branched C₁-C₆ alkyl radicals substituted by at least one radical chosen from hydroxy, C₁-C₄ alkoxy, amino, C₁-C₄ monoalkylamino, and C₁-C₄ dialkylamino radicals,
R′ and R″, which may be identical or different, are chosen from:
C₁-C₆ alkyl radicals,
C₁-C₆ alkoxy radicals,
C₁-C₆ hydroxy-alkoxy radicals,
C₁-C₆ alkoxy(C₁-C₆)alkyl radicals, and
C₁-C₆ mono- and poly-hydroxy alkyl radicals,
R₅ is chosen from:
hydrogen,
linear and branched C₁-C₁₂ alkyl radicals,
tosyl radicals,
mesyl radicals, and
benzene rings optionally functionalized with at least one group chosen from C₁-C₄ alkyl groups and 3-carboxypropyl groups, and
n and m, which may be identical or different, are chosen from integers ranging from 0 to 4.

23. A multi-compartment kit comprising at least one first compartment containing at least one dyeing composition and at least one second compartment containing at least one oxidizing agent,
wherein the at least one dyeing composition comprises, in a medium that is suitable for dyeing, at least one para-phenylenediamine oxidation base chosen from compounds of formula (I) and the addition salts thereof: 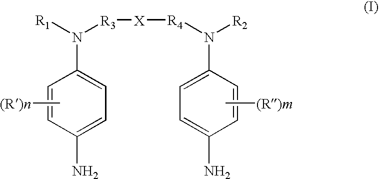
wherein:
X is chosen from:
sulphur, and
—NR₅— radicals,
R₃ and R₄, which may be identical or different, are chosen from C₂-C₆ alkylene radicals,
R₁ and R₂, which may be identical or different, are chosen from:
hydrogen,
linear and branched C₁-C₆ alkyl radicals, and
linear and branched C₁-C₆ alkyl radicals substituted by at least one radical chosen from hydroxy, C₁-C₄ alkoxy, amino, C₁-C₄ monoalkylamino, and C₁-C₄ dialkylamino radicals,
R′ and R″, which may be identical or different, are chosen from:
C₁-C₆ alkyl radicals,
C₁-C₆ alkoxy radicals,
C₁-C₆ hydroxy-alkoxy radicals,
C₁-C₆ alkoxy(C₁-C₆)alkyl radicals, and
C₁-C₆ mono- and poly-hydroxy alkyl radicals,
R₅ is chosen from:
hydrogen,
linear and branched C₁-C₁₂ alkyl radicals,
tosyl radicals,
mesyl radicals, and
benzene rings optionally functionalized with at least one group chosen from C₁-C₄ alkyl groups and 3-carboxypropyl groups, and
n and m, which may be identical or different, are chosen from integers ranging from 0 to 4.

24. A process for the synthesis of a para-phenylenediamine of formula (I): 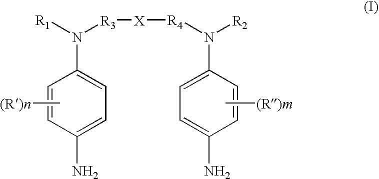
comprising reducing a nitrogen-containing compound of formula (II): 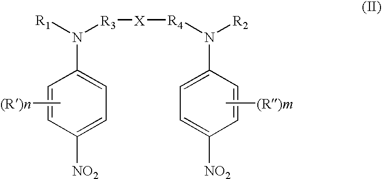
wherein in formulas (I) and (II):
X is chosen from:
sulphur, and
—NR₅— radicals,
R₃ and R₄, which may be identical or different, are chosen from C₂-C₆ alkylene radicals,
R₁ and R₂, which may be identical or different, are chosen from:
hydrogen,
linear and branched C₁-C₆ alkyl radicals, and
linear and branched C₁-C₆ alkyl radicals substituted by at least one radical chosen from hydroxy, C₁-C₄ alkoxy, amino, C₁-C₄ monoalkylamino, and C₁-C₄ dialkylamino radicals,
R′ and R″, which may be identical or different, are chosen from:
C₁-C₆ alkyl radicals,
C₁-C₆ alkoxy radicals,
C₁-C₆ hydroxy-alkoxy radicals,
C₁-C₆ alkoxy(C₁-C₆)alkyl radicals, and
C₁-C₆ mono- or poly-hydroxy alkyl radicals,
R₅ is chosen from:
hydrogen,
linear and branched C₁-C₁₂ alkyl radicals,
tosyl radicals,
mesyl radicals, and
benzene rings optionally functionalized with at least one group chosen from C₁-C₄ alkyl groups and 3-carboxypropyl groups, and
n and m, which may be identical or different, are chosen from integers ranging from 0 to 4.

25. A nitrogen-containing compound of formula (II): 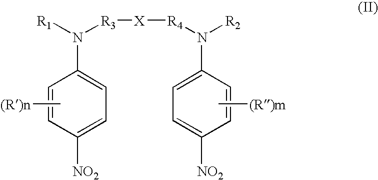
wherein:
X is chosen from:
sulphur, and
—NR₅— radicals,
R₃ and R₄, which may be identical or different, are chosen from C₂-C₆ alkylene radicals,
R₁ and R₂, which may be identical or different, are chosen from:
hydrogen,
linear and branched C₁-C₆ alkyl radicals, and
linear and branched C₁-C₆ alkyl radicals substituted by at least one radical chosen from hydroxy, C₁-C₄ alkoxy, amino, C₁-C₄ monoalkylamino, and C₁-C₄ dialkylamino radicals,
R′ and R″, which may be identical or different, are chosen from:
C₁-C₆ alkyl radicals,
C₁-C₆ alkoxy radicals,
C₁-C₆ hydroxy-alkoxy radicals,
C₁-C₆ alkoxy(C₁-C₆)alkyl radicals, and
C₁-C₆ mono- and poly-hydroxy alkyl radicals,
R₅ is chosen from:
hydrogen,
linear and branched C₁-C₁₂ alkyl radicals,
tosyl radicals,
mesyl radicals, and
benzene rings optionally functionalized with at least one group chosen from C₁-C₄ alkyl groups and 3-carboxypropyl groups, and
n and m, which may be identical or different, are chosen from integers ranging from 0 to 4,
with the proviso that the compound of formula (II) is not chosen from the following compounds: [chemical structure]
